(19) United States
(12) Patent Application Publication
(10) Pub. No.: US 2015/0020813 A1
(43) Pub. Date: Jan. 22, 2015

(54) ANESTHESIA MACHINE COVER WITH POCKETS AND METHOD OF USE THEREOF

(71) Applicant: ADITI HOLDINGS LLC, Miami, FL (US)

(72) Inventors: Murlikrishna KANNAN, Miami, FL (US); Sushmitha SANTHOSH, Miami, FL (US)

(21) Appl. No.: 14/334,085

(22) Filed: Jul. 17, 2014

Related U.S. Application Data
(60) Provisional application No. 61/847,206, filed on Jul. 17, 2013.

(51) Int. Cl. A61B 19/08; A61B 19/12; A61B 19/10; A61M 16/01

(52) U.S. Cl. CPC A61B 19/081; A61M 16/01; A61B 19/12; A61B 19/10; A61B 19/088; A61B 2019/086; A61B 2019/085
USPC 128/852; 128/849; 128/856; 128/855; 128/853

(57) ABSTRACT

The present invention is an anesthesia machine cover and method intended to address the dual problems of cleanliness and organization of anesthesia equipment during surgery and is intended for single patient use. The device covers anesthesia equipment quickly prior to surgery and prevents contamination while providing for better containment and organization of ancillary equipment used for each procedure. The cover is easily removable for disposal post procedure.

### CROSS-REFERENCE TO RELATED APPLICATIONS

This application claims priority to provisional patent application number 61/847,206 filed on Jul. 17, 2013, the contents of which are expressly incorporated by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The anesthesia environment is an area with which the anesthesia provider constantly interacts. The Anesthesia environment, generally consists of one or more cart mounted pieces of equipment for administering anesthesia and for monitoring patient vital signs. In most cases, will consist of an anesthesia machine. The anesthesia machine serves two important functions; 1) it allows administration of anesthetic gases and 2) it provides a place set patient care equipment on the machine.

The anesthesia machine is a complex electronic instrument. The machine allows the anesthesia provider to administer oxygen and anesthetic gases in precise concentration. The precise administration of oxygen and anesthetic gases is achieved by operating knobs and buttons that activate flow control systems.

The anesthesia machine provides the anesthesia provider a place to set equipment that will be used during patient care. This consists of monitoring equipment and equipment that are inserted into the patient like endotracheal tubes, airways, laryngoscopes etc. Equipment inserted into the patient is frequently placed back on the anesthesia machine.

### THE PROBLEM

The anesthesia machine is the most important resource for the anesthesia provider. The machine has undergone several modifications since its introduction in the early 1900s to improve the safety profile. Modern anesthesia machines have touch screens and advanced electronics.

Regardless of the make and type of the anesthesia machine, the anesthesia provider will place equipment that will be needed on the anesthesia machine. These include laryngoscopes, airways, endotracheal tubes, and intravenous kits to name a few. Some of these devices are frequently placed back on the machine after use during patient care. Common interventions performed by anesthesia providers involve insertion of airway devices in the patient's mouth, insertion of tubes into the patient stomach, and insertion of intravenous lines. Whilst performing these interventions, the anesthesia provider is constantly touching the anesthesia machine to make adjustments. As a result the anesthesia providers frequently contaminate the anesthesia machine with blood and body fluids.

Current practice follows guidelines that recommend that medical equipment surfaces be cleaned by disinfecting wipes after patient care. This involves complete top down cleaning of the machine each time and every time between surgeries. This involves time consuming meticulous cleaning which increases operating room turn over times, as well as the use of harmful chemicals and disinfectants on electronic equipment not to mention the need for expensive human labor.

Multidrug resistant bacteria like Methicillin resistant staphylococci, Clostridium difficile, Vancomycin resistant enterococci are all a major threat to patient safety. Patients infected with these deadly microbes are labeled “contact isolation patients”. These patients need surgery from time to time and they are brought into the operating room. Different institutions have different policies to deal with contact isolation patients in the operating room. The one common objective is to reduce the transfer and spread of these dangerous microbes to other patients. This involves extensive cleaning of the operating room and the equipment in the operating room. Inadequate cleaning of the anesthesia machine increases risk of cross contamination.

Multiple studies have shown the presence of blood on the anesthesia machines after cleaning Infection causing bacteria ranging from simple bacteria to multidrug resistant bacteria has been isolated from the surface of the anesthesia machines. In 2008, Loftus et al, proved that bacteria from one patient is transmitted to the anesthesia machine which is then transmitted to the intravenous ports of the next patient. This conclusively proved that current practices may be inefficient and dangerous to patients. There is a growing consensus that these cleaning procedures must be improved.

The safe harbor, transport of contaminated anesthesia equipment gets very little attention. Anesthesia equipment is contaminated with patient body fluids like saliva, blood and sputum. Currently, contaminated equipment is stored in a corner of the operating room in close proximity to clean equipment. Depending on institutional policy, this equipment is removed at the end of the case or at the end of the day. In either case, this is an unsafe way to handle equipment with biohazard contamination, placing both patients and healthcare providers at risk.

Yet another problem that is infrequently addressed is the setting up of anesthesia equipment for the next patient right next to the contaminated equipment. Experts have discouraged this practice primarily because the anesthesia environment is a contaminated environment. A clean setup cannot be accomplished in a contaminated environment. There are different reasons for this practice but the most common has been to reduce operating room turnover times between cases. Contaminated equipment sometimes is not removed and is discovered during subsequent patient care.

### SUMMARY OF THE INVENTION

The Anesthesia machine cover is intended to address the dual problems of cleanliness and organization of equipment during anesthesia care of the patient. The cover is a single patient use, easily applied cover that can be applied

The cover is intended as a barrier to protect the machine. Barrier concept uses the simple idea of prevention is better than cure. Barrier concept has been used for ages in condoms. Efficient barriers have revolutionized surgical gowns and drapes. A good surgical barrier is a minimum requirement for surgery as this prevents infection more than anything else.

The cover is a simple non-sterile barrier that will run along the entire working surface of the machine. The cover will allow the continued operation of the anesthesia machine but does not require direct contact to the machine. The barrier will be made according to industry guidelines to be electrostatic, fire retardant and tear resistant. This barrier will prevent biohazard material like blood and patient body fluids from contaminating the anesthesia machine during patient care.

This protection minimizes cleaning requirements of the anesthesia machine with disinfectant wipes after surgery.

The second important function of the cover is the safe organization and containment of contaminated anesthesia equipment during patient care. The cover allows the anesthesia provider the ability and location to place clean and contaminated devices on the anesthesia machine with the knowledge that the barrier is clean and the barrier will protect the anesthesia machine from contaminated equipment. This allows the provider to focus on the patient and not worry about potentially contaminating multiple surfaces in the anesthesia environment.

The cover has pouches or receptacles to house contaminated instruments. These pouches are leak free and may be detachable from the cover. At the end of surgery, the pouches that harbor reusable equipment like a fiber-optic scope can be safely detached from the body of the cover and stay with the instrument while transported to other sites where they may be cleaned and sterilized for next patient use.

The present invention will reduce clutter. It will result in better organization of equipment, safe harbor of equipment and safe transport of contaminated equipment.

It is an object of the invention to provide customizable barrier protection for anesthesia machines.

It is an object of the invention to protect the anesthesia machine from contamination.

It is an object of the invention to reduce anesthesia machine turnover time in between surgical cases.

It is an object of the invention to provide a means of covering each component of an anesthesia machine

### BRIEF DESCRIPTION OF THE DRAWINGS

FIG. 1 is an isometric view of an anesthesia machine to which a covering might be applied.

FIG. 2a is an isometric view of an anesthesia machine cover.

FIG. 2b is an isometric view of an anesthesia machine cover held in place with straps.

FIG. 3 is a view of an anesthesia machine covered by the present invention

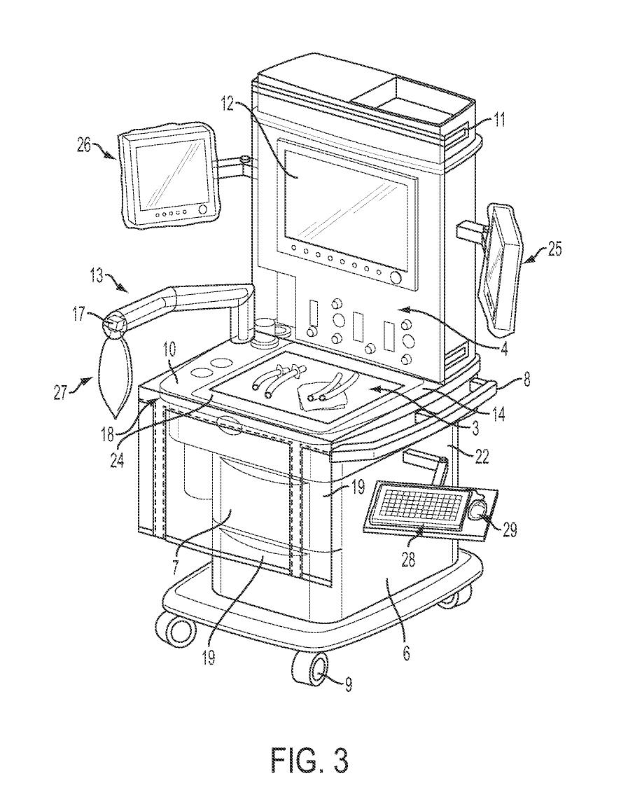

### DETAILED DESCRIPTION OF THE INVENTION

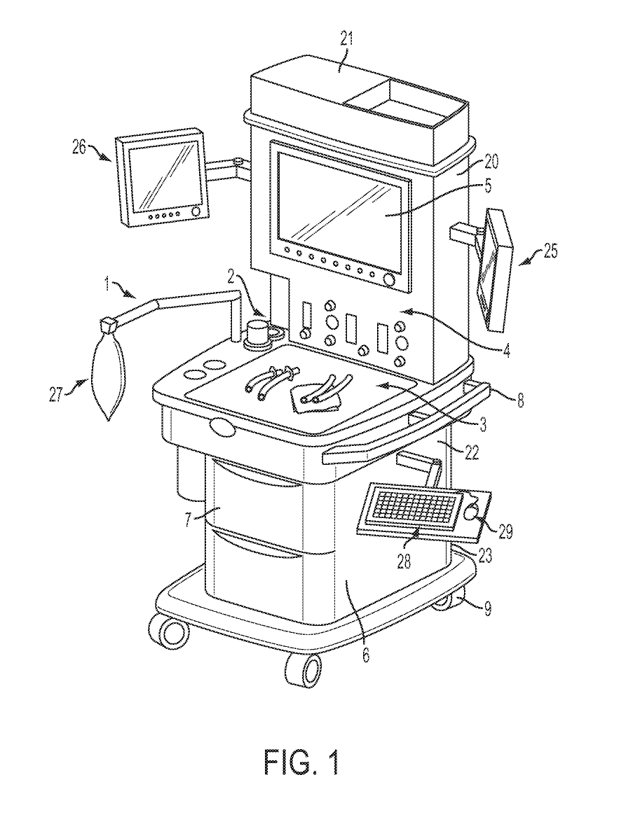

FIG. 1 shows an isometric view of an anesthesia machine 5 resting on a cart 6. In some instances the cart includes an arm 1 attached to the cart 6 at a proximal end having a distal end for holding cables and/or tubes 17 which are connected to the cart 6. Most commonly the arm delivers the anesthesia gas. An insufflation bag 27 is also attached to the arm.

The anesthesia machine comprises a vertical surface 4 having a plurality of indicators and control knobs 2 which are used to monitor the delivery of anesthetics and patient vital signs. The indicators and knobs must be accessible and visible during any procedure.

The cart 6 includes a horizontal work surface 3 and may optionally include drawers 7, castors or wheels 9 and rails 8 for mounting additional equipment. The horizontal work surface 3 is generally used to hold the most commonly needed equipment to set up and deliver anesthesia for a given procedure. This might include laryngoscopes with different blades, tracheal tubes, preloaded syringes with medications, facemasks of different sizes, oral airways, nasopharyngeal airways, gauze, intravenous kits, fibreoptic bronchoscope and other needed equipment depending on complexity of surgical case.

The anesthesia machine 5 may include at least one video monitor 12 for displaying patient information and/or running software. The video monitor may be mounted on the surface of anesthesia machine or may be mounted on brackets or arms attached to the anesthesia machine or cart. A computer keyboard 28 and/or a mouse 29 may be present.

Anesthesia machines are by necessity set up next to an operating table. As is readily apparent, when present in an operating room environment the anesthesia machine is subject to contamination by blood and other body fluids. Endotracheal tubes are frequently placed on the cart before insertion or after removal. These tubes are regularly contaminated with sputum, blood and other body secretion and fluids, which get transferred to the anesthesia machine and/or the cart. Further the anesthesia provider, will by necessity touch the patient prepping them for surgery, during surgery and during removal of any equipment post-surgery. The anesthesia machine will by necessity by contaminated by the need to touch it during the procedure.

While procedures exist for cleaning and disinfecting the anesthesia machine and the cart upon which it rests, the procedures rarely remove 100% of the biological contamination. This is due to both the complexity of the surfaces to be cleaned and the fact that there is often pressure to prepare an operating room for the next patient. As such, anesthesia equipment is presently a likely means for transferring pathogens between patients. Hospital acquired infections A failsafe means is needed to prevent and stop this contamination.

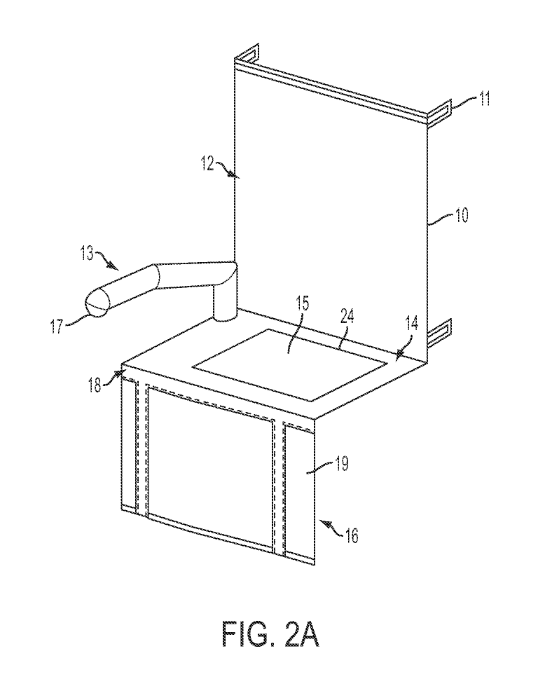

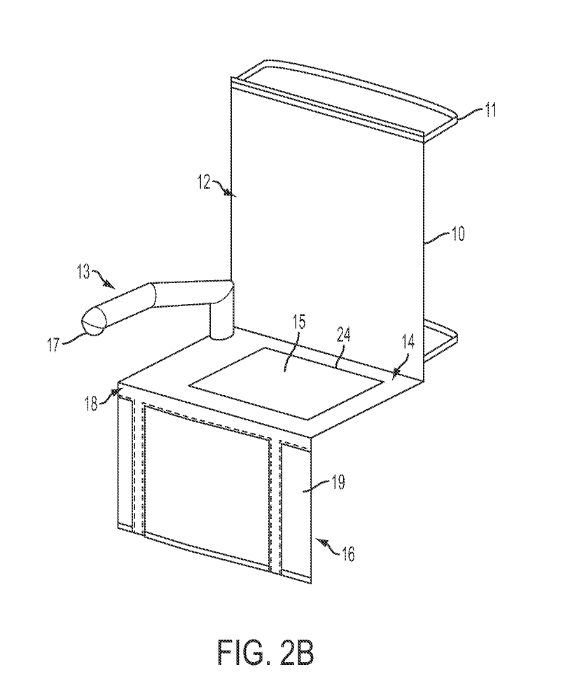

Referring to FIG. 2, the present invention comprises an anesthesia cover 10 which protects the anesthesia machine 5 and cart 6 from contamination. The cover 10 is made from any material which is suitable for use in the operating room such as plastics or fabrics which are either cheap enough to be disposable after use or can be sterilized using existing equipment. Most preferred are disposable plastics such as high density polyethylene, although other plastic sheeting may be used.

The cover 10 comprises at least a vertical surface cover 12 for covering the vertical face of the anesthesia machine 5 and a horizontal surface cover 15 for covering the horizontal surface 3 of the cart 6. Optionally, the cover 10, further comprises a second vertical surface cover 19 for covering the front 7 of the cart 6. The cover 10 may optionally include additional coverage for the sides and back of the anesthesia machine 20, 21 and sides and back of the cart 22, 23. Optionally, one or more pockets 16 may be included in the cover 10. The pockets may be removable at perforations 18.

In some embodiments, the cover optionally includes at least one sleeve 13 for covering an arm 1 on the cart 6. The sleeve 13 may include a means for closing the opening at end 17 around the arm 1, such means comprising an elastic opening or drawstring, adhesive, tape, clamps, zip ties, elastic bands, etc. Such sleeves 13 may also be used to cover tubing (Not shown) from the anesthesia machine 5. The sleeve 13 may be integrated into the cover 10 or it may be a separate piece. If the sleeve is a separate piece, it may be secured to the cover with any suitable means. In some embodiments the joint may contain an adhesive or include tape to better secure where the cover 10 and sleeve meet to prevent the anesthesia machine and cart from being contaminated.

The cover 10 should either be of a sufficient optical clarity to allow observation of the monitors and or meters on the anesthesia machine 5 or have clear portions for viewing the instrumentation. The material of the cover 10 should not be so thick that a gloved anesthetist cannot operate switches or knobs. When polyethylene sheeting is used the material is preferably from 1-5 mils thick with 2-3 mils most preferred. One of skill in the art will be able to choose suitable materials based on their resistance to tearing or perforation and the tactile feel of knobs and switches when contacted through the material.

The cover 10 may be attached to the anesthesia machine 5 and cart 10 using various means such as ties, Velcro, adhesive, tape, rubber bands, straps, thread, zippers, hooks, buttons, rivets or other mechanical fasteners.

In some embodiments the cover is packaged with covers for other equipment such as additional monitors 24 and 25, keyboards 28, computer mice 29, laryngoscopes, etc. Such covers are suitably form fitting for the device they are covering. In one embodiment such covers are a form fitting bag which can be snugged around the device. In others, they are sized sheets of plastic which may be draped or folded around the device and secured. In one embodiment, the cover for a computer monitor is a plastic sheet with elastic around the perimeter allowing the device to be secured to the monitor with the elastic.

The cover 10 may be manufactured such that it is made from a stretchable material to better form fit. Elastic may be used as needed to allow material to stretch for placement and contract to hold the covers in place.

In one embodiment, the cover 10, consists of a vertical surface cover 12 which is sized large enough to wrap around the top 21 and sides 20 of the anesthesia machine 5, a horizontal surface cover 15 which is sized to drape down the sides 23 of the cart 6, and a second vertical surface cover 19 which covers the front 7 of the cart 6.

Optionally the cover 10 includes a horizontal pad 24 which is placed on or integrated into the horizontal cover 5. The horizontal pad is made of a material intended to provide a more robust and or less slippery surface for placing items, papers, or a note pad and pen used during a procedure. The underside may also be provided with a tacky, sticky, or peel and stick material to prevent slippage or movement of this portion of the drape. In one embodiment the pad 24 is made of paper or paper like material.

Pouches 16 can be located on any surface where they would not interfere with reading the instrumentation or manipulating the switches or knobs. Most preferred is along the second vertical surface cover 19. The pouch(es) or receptacle(s) are intended to hold elongated, tethered, electrically, pneumatically, or optically connected instruments or tubes and is intended to keep them from touching other surfaces or instruments and to keep them organized and prevent tangling. Additionally a trash receptacle may be provided. The pouch(es) are formed into the cover but may be separable by perforations 18, by cutting, or any similar such method to facilitate removal of one or more pouches 18, such as for the removal and transport or disposal of a contaminated tool or instrument. In other embodiments, the pouches 16 are attached with adhesive, tape or Velcro.

In another embodiment, the cover 10 is wrapped around the anesthesia machine 5 and cart 6 like a skirt.

Optionally, the cover may include one or more perforated sections 30 which open to allow access directly to the anesthesia machine.

Optionally, the cover may be coated with, or impregnated with a disinfectant, antiseptic, antibiotic or an antimicrobial composition during manufacturing or during installation. Such compositions could include triclosan, or anything suitable for use in the operating room environment.

It will be readily apparent from one of skill in the art that the present cover 10 can be comprised of a single part or multiple pieces which can attach to each other, or not.

The present invention further comprises a method of covering an anesthesia machine to protect it from contamination and for protecting patients from contamination emanating from the anesthesia machine. The method comprises:

a. Placing an open cover 10 on an anesthesia machine 5.

b. Attaching the top of the cover to the top of the anesthesia machine

c. Attaching the cover to the horizontal surface of the anesthesia cart

d. Placing the lower vertical surface across the front of the anesthesia cart.

e. placing a plastic sleeve over the arm of the cart 7

f. Optionally applying an antimicrobial composition to the cover and /or any exposed parts of the anesthesia machine or cart.

g. Using the anesthesia machine 6 through the cover 10 during the procedure.

h. Optionally placing any contaminated equipment into the optional pouches on the front of the cover.

i. Optionally, separating the pouches to allow transport of the contaminated equipment.

j. Remove the arm cover at the completion of the procedure

k. Remove and discard the cover from the anesthesia machine and cart.

In a preferred embodiment the cover is folded in such a way that it can be installed as follows:

a. The package is opened and the cover initially unfolded on the horizontal surface of the cart.

b. A portion of the cover for covering the anesthesia machine is folded upwards over the anesthesia machine and secured in place.

c. A portion of the cover for covering the cart is unfolded downwards and secured in place.

d. The arm cover is placed in position and any additional monitor, keyboard, mouse or any other instrument covers are positioned in place.

It will be readily apparent to one of skill in the art that substantial deviation from the disclosure herein is possible without departing from the spirit of the invention.

1. An anesthesia machine cover comprising:
at least one vertical cover for covering a vertical surface of an anesthesia machine or cart;
at least once horizontal surface cover for covering a horizontal of the anesthesia machine or cart;
a means for securing the cover to the anesthesia machine and/or cart;
wherein the cover allows visibility through the cover to gauges and or indicators; and
wherein the cover does not substantially interfere with the users ability to actuate buttons, knobs, and or switches as required during a procedure.

2. The anesthesia machine cover of claim 1 which includes an arm cover.

3. The anesthesia machine cover of claim 2 wherein the arm cover is a separate sleeve.

4. The anesthesia machine cover of claim 2 wherein the arm cover is integrated into the anesthesia machine cover.

5. The anesthesia machine cover of claim 1 which includes at least one pouch to hold an instrument and or for the disposal of trash.

6. The anesthesia machine cover of claim 1 which includes an attachment component to affix the cover to a vertical surface and or to prevent movement on a horizontal surface.

7. The anesthesia machine cover of claim 6 wherein the attachment component comprises adhesive tapes, Velcro, elastic straps, snaps, buttons, and/or rubber bands.

8. The anesthesia machine cover of claim 6 wherein the attachment component comprises adhesive tape.

9. The anesthesia machine cover of claim 1 wherein the cover has at least one perforation to allow tearing the cover for direct access to the anesthesia machine and/or cart or for removal of a pouch.

10. A method of reducing contamination of an anesthesia machine comprising:
covering an anesthesia machine and/or anesthesia cart with a cover which comprises a covering portion for at least one vertical and/or horizontal surface of the machine while allowing visibility through the cover to gages and indicators as well the ability to actuate buttons, knobs, and/or switches required during a procedure; and securing the hygienic cover to the machine.

11. The method of claim 10 wherein at least one additional cover is placed on portions of the anesthesia machine not readily covered by the prefolded cover.

12. The method of claim 11 where in the additional covers are selected from an arm cover, monitor cover, keyboard cover, mouse cover or instrument cover.

13. The method of claim 10 which includes the cover includes at least once pouch and the user places contaminated instruments into the pouch for transport or disposal.

14. The method in claim 10 in which the cover further comprises a means of separating the holding component or pouch from body of the cover.

15. The method of claim 10 further comprising placing a separate horizontal pad on a horizontal surface for better holding instruments.

16. The method of claim 10 comprising: providing a pre-folded cover which is placed on a horizontal surface of the anesthesia machine; unfolding a portion of the cover in a first vertical direction to cover a first vertical portion of the anesthesia machine; unfolding a second vertical direction to cover a second vertical portion of the anesthesia machine.

17. The method of claim 13 wherein the pouch is attached with perforations or a removable adhesive.

18. The method of claim 10, wherein the cover has at least one perforation or opening to allow direct access to the anesthesia machine.

19. An kit for covering an anesthesia machine cover comprising:
an anesthesia machine cover having at least one covering portion for a horizontal surface and at least one covering portion for a vertical surface; and
at least one additional cover for a portion of the anesthesia set up not readily covered by the anesthesia machine cover.

20. The kit of claim 15 wherein the additional cover is selected from an arm cover, monitor cover, keyboard cover, mouse cover or instrument cover.
